(19) United States
(12) Patent Application Publication
(10) Pub. No.: US 2022/0193074 A1
(43) Pub. Date: Jun. 23, 2022

(54) COMPOUNDS WITH ANTI-TUMOR ACTIVITY AGAINST CANCER CELLS BEARING TYROSINE KINASE INHIBITOR RESISTANT EGFR MUTATIONS

(71) Applicant: Board of Regents, The University of Texas System, Austin, TX (US)

(72) Inventors: Jacqulyne ROBICHAUX, Houston, TX (US); Monique NILSSON, Houston, TX (US); John V. HEYMACH, Houston, TX (US)

(73) Assignee: Board of Regents, The University of Texas System, Austin, TX (US)

(21) Appl. No.: 17/604,689

(22) PCT Filed: Apr. 16, 2020
(86) PCT No.: PCT/US2020/028547

§ 371 Date: Oct. 18, 2021

Related U.S. Application Data
(60) Provisional application No. 62/835,354, filed on Apr. 17, 2019.

(51) Int. Cl. A61K 31/506; A61K 31/517; A61K 31/519; A61K 31/4706; A61K 31/5377; A61K 31/496; A61K 31/55; A61P 35/00

(52) U.S. Cl. CPC A61K 31/506; A61K 31/517; A61K 31/519; A61P 35/00; A61K 31/5377; A61K 31/496; A61K 31/55; A61K 31/4706

(57) ABSTRACT

The present disclosure provides methods of treating cancer in a patient determined to have osimertinib resistant EGFR mutations by administering a second-generation quinazolinamine derivative tyrosine kinase inhibitor.

This application claims the benefit of U.S. Provisional Patent Application No. 62/835,354, filed Apr. 17, 2019, the entirety of which is incorporated herein by reference.

### BACKGROUND

### 1. Field

The present invention relates generally to the field of molecular biology and medicine. More particularly, it concerns methods of treating patients with tyrosine kinase inhibitor resistant EGFR mutations.

### 2. Description of Related Art

Approximately 10% of non-small-cell lung cancers (NSCLC) have epidermal growth factor receptor (EGFR) mutations resulting in increased sensitivity to tyrosine kinase inhibitors (TKIs) such as gefitinib erlotinib, and osimertinib. Recently osimertinib has been approved for the first line setting for EGFR mutant NSCLC4, but de novo resistance and acquired resistance are still a therapeutic obstacle for many patients. A series of atypical and acquired EGFR mutations may potentially confer osimertinib resistance. Studies have shown that these atypical and acquired resistance mutations change the confirmation of the drug binding pocket near the solvent front of osimertinib causing changes in the binding affinity of the drug to the receptor. Thus, there is an unmet need for novel therapies for treating resistant EGFR mutant cancers.

### SUMMARY

Embodiments of the present disclosure provides methods and compositions for treating cancer in patients with resistant EGFR mutations. In a first embodiment, there is provided a method of treating cancer in a subject comprising administering an effective amount of a quinazolinamine derivative tyrosine kinase inhibitor (TKI) to the subject, wherein the subject has been determined to have one or more epidermal growth factor receptor (EGFR) TKI resistant mutations. In some aspects, the patient is human.

In certain aspects, the quinazolinamine derivative TKI is a covalently binding quinazolinamine TKI. In some aspects, the covalently binding quinazolinamine TKI is afatinib, tarloxotinib-TKI, dacomitinib, pelitinib, or allitinib. In certain aspects, the quinazolinamine derivative TKI is a non-covalently binding quinazolinamine TKI. In some aspects, the non-covalently binding quinazolinamine TKI is sapatinib, AZD3759, Varlitinib, TAK-285, or gefitinib. In certain aspects, the quinazolinamine derivative TKI is formulated as a tablet.

In certain aspects, the one or more EGFR TKI resistant mutations comprise a point mutation, insertion, and/or deletion of 1-18 nucleotides at exon 18, 19, 20, or 21. In some aspects, the one or more EGFR TKI resistant mutations comprise one or more point mutations, insertions, and/or deletions of 3-18 nucleotides between amino acids 688-728 of exon 18. In particular aspects, the one or more EGFR exon 18 mutations are located at one or more residues selected from the group consisting of E709, L718, G719, S5720, and G724. In specific aspects, the one or more EGFR exon 18 mutations comprise E709A, E790K, L718Q, L718V, G719A, G719S, S720P, and/or G724S. In some aspects, the one or more EGFR TKI resistant mutations comprise one or more point mutations, insertions, and/or deletions of 3-18 nucleotides between amino acids 729-761 of exon 19. In certain aspects, the one or more EGFR exon 19 mutations are located at one or more residues selected from the group consisting of 1744, L747, L747, A755, K757, and/or D761. In particular aspects, the one or more EGFR exon 19 mutations comprise I744V, I744T, L747S, L747FS, A755T, K757R, and/or D761N. In some aspects, the one or more EGFR TKI resistant mutations comprise one or more point mutations, insertions, and/or deletions of 3-18 nucleotides between amino acids 763-823 of exon 20. In certain aspects, the one or more EGFR exon 20 mutations are located at one or more residues selected from the group consisting of A763, S768, V769, H773, D770, V774, C775, S784, L792, G796, C797, S811, and R776. In some aspects, the one or more EGFR exon 20 mutations comprise D770insNPG, S784F, R776C, S768I, V774M, S768I, H773insAH, H773insNPH, V774A, V769L, V769M, S768dupSVD, A763insLQEA, L792H, G796D, G796S, S784F, C775Y and/or S811F. In certain aspects, the one or more EGFR TKI resistant mutations comprise one or more point mutations, insertions, and/or deletions of 3-18 nucleotides between amino acids 824-875 of exon 21. In specific aspects, the one or more EGFR exon 21 mutations are located at one or more residues selected from the group consisting of, L833, V834, G836, V843, T854, L861, L861, L862, L844 and L858. In some aspects, the one or more EGFR exon 21 mutations may comprise L833F, V834L, L858R, L861Q, V843I, L861R, L862V, L844V, L861Q, G836S, and/or T854I. In some aspects, the subject has been determined to have 2, 3, or 4 EGFR TKI resistant mutations. In certain aspects, the one or more EGFR TKI resistant mutations are at residues E709, L718, G719, G724, C797, V843, T854, L861, and/or L792. In some aspects, the subject has been determined to not have an EGFR mutation at residue C797 or T790. In particular aspects, the subject is determined to not have an EGFR mutation at residue T790. In other aspects, the subject is determined to have a T790 mutation alone or in combination with another mutation. In certain aspects, the subject is determined to have a mutation at residue at C797. In some aspects, the one or more EGFR TKI resistant mutations are selected from the group consisting of G719X, E709X, G724S, L718X, L861Q, T8541, V8431, C797S, and/or L792X, wherein X is any amino acid. In particular aspects, the one or more EGFR TKI resistant mutations are selected from the group consisting of L861Q, G719S, L858R/L792H, L858R/C797S, and Exl9del/C797S.

In some aspects, the subject has been previously administered a TKI. In certain aspects, the subject is resistant to the previously administered TKI. In some aspects, the TKI is lapatinib, afatinib, dacomitinib, osimertinib, ibrutinib, nazartinib, olmutinib, rociletinib, naquotinib or neratinib. In particular aspects, the TKI is osimertinib, ibrutinib, nazartinib, olmutinib, rociletinib, or naquotinib. In specific aspects, the TKI is osimeritinib.

In certain aspects, the subject was determined to have an EGFR TKI resistant mutation by analyzing a genomic sample from the patient. In some aspects, the genomic sample is isolated from saliva, blood, urine, normal tissue, or tumor tissue. In certain aspects, the presence of an EGFR TKI resistant mutation is determined by nucleic acid sequencing or PCR analyses.

In some aspects, the quinazolinamine derivative TKI is administered orally. In certain aspects, the quinazolinamine derivative TKI is administered daily. In particular aspects, the quinazolinamine derivative TKI is administered on a continuous basis. In some aspects, the quinazolinamine derivative TKI is administered on 28 day cycles.

In additional aspects, the method further comprises administering an additional anti-cancer therapy. In some aspects, the additional anti-cancer therapy is chemotherapy, radiotherapy, gene therapy, surgery, hormonal therapy, anti-angiogenic therapy or immunotherapy. In particular aspects, the poziotinib and/or anti-cancer therapy are administered intravenously, subcutaneously, intraosseously, orally, transdermally, in sustained release, in controlled release, in delayed release, as a suppository, or sublingually. In some aspects, administering the poziotinib and/or anti-cancer therapy comprises local, regional or systemic administration. In particular aspects, the poziotinib and/or anti-cancer therapy are administered two or more times.

In some aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In particular aspects, the cancer is non-small cell lung cancer.

Further provided herein is a pharmaceutical composition comprising quinazolinamine derivative TKI for use in a subject determined to have one or more EGFR TKI resistant mutations. In some aspects, the composition is further defined as an oral composition. In certain aspects, the composition comprises 5-25 mg of a quinazolinamine derivative TKI. In some aspects, the composition is formulated as a tablet. In some aspects, the subject is being treated with an anti-cancer therapy.

In certain aspects, the one or more EGFR TKI resistant mutations comprise a point mutation, insertion, and/or deletion of 1-18 nucleotides at exon 18, 19, 20, or 21. In some aspects, the one or more EGFR TKI resistant mutations comprise one or more point mutations, insertions, and/or deletions of 3-18 nucleotides between amino acids 688-728 of exon 18. In particular aspects, the one or more EGFR exon 18 mutations are located at one or more residues selected from the group consisting of E709, L718, G719, S5720, and G724. In specific aspects, the one or more EGFR exon 18 mutations comprise E709A, L718Q, L718V, G719A, G719S, S720P, and/or G724S. In some aspects, the one or more EGFR TKI resistant mutations comprise one or more point mutations, insertions, and/or deletions of 3-18 nucleotides between amino acids 729-761 of exon 19. In certain aspects, the one or more EGFR exon 19 mutations are located at one or more residues selected from the group consisting of 1744, L747, L747, A755, K757, and/or D761. In particular aspects, the one or more EGFR exon 19 mutations comprise I744V, I744T, L747S, L747FS, A755T, K757R, and/or D761N. In some aspects, the one or more EGFR TKI resistant mutations comprise one or more point mutations, insertions, and/or deletions of 3-18 nucleotides between amino acids 763-823 of exon 20. In certain aspects, the one or more EGFR exon 20 mutations are located at one or more residues selected from the group consisting of A763, S768, V769, H773, D770, V774, C775, S784, L792, G796, C797, S811, and R776. In some aspects, the one or more EGFR exon 20 mutations comprise D770insNPG, S784F, R776C, S768I, V774M, S768I, H773insAH, H773insNPH, V774A, V769L, V769M, S768dupSVD, A763insLQEA, L792H, G796D, S784F, C775Y and/or S811F. In certain aspects, the one or more EGFR TKI resistant mutations comprise one or more point mutations, insertions, and/or deletions of 3-18 nucleotides between amino acids 824-875 of exon 21. In specific aspects, the one or more EGFR exon 21 mutations are located at one or more residues selected from the group consisting of, L833, V834, G836, V843, T854, L861, L861, L862, L844 and L858. In some aspects, the one or more EGFR exon 21 mutations may comprise L833F, V834L, L858R, L861Q, V843I, L861R, L862V, L844V, L861Q, G836S, and/or T854I. In some aspects, the subject has been determined to have 2, 3, or 4 EGFR TKI resistant mutations. In certain aspects, the one or more EGFR TKI resistant mutations are at residues E709, L718, G719, G724, C797, V843, T854, L861, and/or L792. In some aspects, the subject has been determined to not have an EGFR mutation at residue C797 or T790. In particular aspects, the subject is determined to not have an EGFR mutation at residue T790. In other aspects, the subject is determined to have a T790 mutation alone or in combination with another mutation. In certain aspects, the subject is determined to have a mutation at residue at C797. In some aspects, the one or more EGFR TKI resistant mutations are selected from the group consisting of G719X, E709X, G724S, L718X, L861Q, T8541, V8431, C797S, and/or L792X, wherein X is any amino acid. In particular aspects, the one or more EGFR TKI resistant mutations are selected from the group consisting of L861Q, G719S, L858R/L792H, L858R/C797S, and Exl9del/C797S.

A further embodiment provides a method of predicting a response to a quinazolinamine derivative TKI alone or in combination with a second anti-cancer therapy in a subject having a cancer comprising detecting a EGFR TKI resistant mutation in a genomic sample obtained from said patient, wherein if the sample is positive for the presence of the EGFR TKI resistant mutation, then the patient is predicted to have a favorable response to the quinazolinamine derivative TKI alone or in combination with an anti-cancer therapy. In some aspects, the genomic sample is isolated from saliva, blood, urine, normal tissue, or tumor tissue. In certain aspects, the presence of a HER exon 21 mutation is determined by nucleic acid sequencing or PCR analyses.

In certain aspects, the one or more EGFR TKI resistant mutations comprise a point mutation, insertion, and/or deletion of 1-18 nucleotides at exon 18, 19, 20, or 21. In some aspects, the one or more EGFR TKI resistant mutations comprise one or more point mutations, insertions, and/or deletions of 3-18 nucleotides between amino acids 688-728 of exon 18. In particular aspects, the one or more EGFR exon 18 mutations are located at one or more residues selected from the group consisting of E709, L718, G719, S5720, and G724. In specific aspects, the one or more EGFR exon 18 mutations comprise E709A, L718Q, L718V, G719A, G719S, S720P, and/or G724S. In some aspects, the one or more EGFR TKI resistant mutations comprise one or more point mutations, insertions, and/or deletions of 3-18 nucleotides between amino acids 729-761 of exon 19. In certain aspects, the one or more EGFR exon 19 mutations are located at one or more residues selected from the group consisting of 1744, L747, L747, A755, K757, and/or D761. In particular aspects, the one or more EGFR exon 19 mutations comprise I744V, I744T, L747S, L747FS, A755T, K757R, and/or D761N. In some aspects, the one or more EGFR TKI resistant mutations comprise one or more point mutations, insertions, and/or deletions of 3-18 nucleotides between amino acids 763-823 of exon 20. In certain aspects, the one or more EGFR exon 20 mutations are located at one or more residues selected from the group consisting of A763, S768, V769, H773, D770, V774, C775, S784, L792, G796, C797, S811, and R776. In some aspects, the one or more EGFR exon 20 mutations comprise D770insNPG, S784F, R776C, S768I, V774M, S768I, H773insAH, H773insNPH, V774A, V769L, V769M, S768dupSVD, A763insLQEA, L792H, G796D, S784F, C775Y and/or S811F. In certain aspects, the one or more EGFR TKI resistant mutations comprise one or more point mutations, insertions, and/or deletions of 3-18 nucleotides between amino acids 824-875 of exon 21. In specific aspects, the one or more EGFR exon 21 mutations are located at one or more residues selected from the group consisting of, L833, V834, G836, V843, T854, L861, L861, L862, L844 and L858. In some aspects, the one or more EGFR exon 21 mutations may comprise L833F, V834L, L858R, L861Q, V843I, L861R, L862V, L844V, L861Q, G836S, and/or T854I. In some aspects, the subject has been determined to have 2, 3, or 4 EGFR TKI resistant mutations. In certain aspects, the one or more EGFR TKI resistant mutations are at residues E709, L718, G719, G724, C797, V843, T854, L861, and/or L792. In some aspects, the subject has been determined to not have an EGFR mutation at residue C797 or T790. In particular aspects, the subject is determined to not have an EGFR mutation at residue T790. In other aspects, the subject is determined to have a T790 mutation alone or in combination with another mutation. In certain aspects, the subject is determined to have a mutation at residue at C797. In some aspects, the one or more EGFR TKI resistant mutations are selected from the group consisting of G719X, E709X, G724S, L718X, L861Q, T8541, V8431, C797S, and/or L792X, wherein X is any amino acid. In particular aspects, the one or more EGFR TKI resistant mutations are selected from the group consisting of L861Q, G719S, L858R/L792H, L858R/C797S, and Exl9del/C797S.

A further embodiment provides a favorable response to the quinazolinamine derivative TKI alone or in combination with an anti-cancer therapy comprises reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, or increased patient survival.

In additional aspects, the method further comprises administering the quinazolinamine derivative TKI alone or in combination with a second anti-cancer therapy to said patient predicted to have a favorable response. In some aspects, the quinazolinamine derivative TKI is administered orally. In certain aspects, the quinazolinamine derivative TKI is administered at a dose of 5-25 mg. In some aspects, the quinazolinamine derivative TKI is formulated as a tablet.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D: Second generation quinazoline based TKIs are more potent and selective than third generation TKIs such as osimertinib in atypical EGFR mutations in vitro. (FIG. 1A) Heatmap of Log IC₅₀ values of Ba/F3 cells expressing primary atypical mutations spanning exons 18-21 treated with indicated inhibitors for 72 hours. Mutations are ordered from most resistant to most sensitive top to bottom, and drugs are ordered from lowest IC₅₀ value to highest IC₅₀ value left to right. (FIG. 1B) Heatmap of ratio of the IC₅₀ values of Ba/F3 cells expressing primary atypical mutations spanning exons 18-21 divided by the IC₅₀ values of Ba/F3 cells expressing WT EGFR (+10 ng/ml EGF) treated with indicated inhibitors for 72 hours. Order of mutations was kept the same from panel A, but drugs were re-ordered from left to right for most selective to least selective. (FIG. 1C) Bar graphs of mean±SEM IC₅₀ values of Ba/F3 cells expressing atypical mutations separated by drug class. N=40 cell lines, and symbols are representative of each cell line. Statistical significances were determined by ANOVA. (FIG. 1D) Bar graphs of mean±SEM Mutant/WT EGFR ratio of Ba/F3 cells expressing atypical mutations separated by drug class. N=40 cell lines, and symbols are representative of each cell line. Statistical significances were determined by ANOVA.

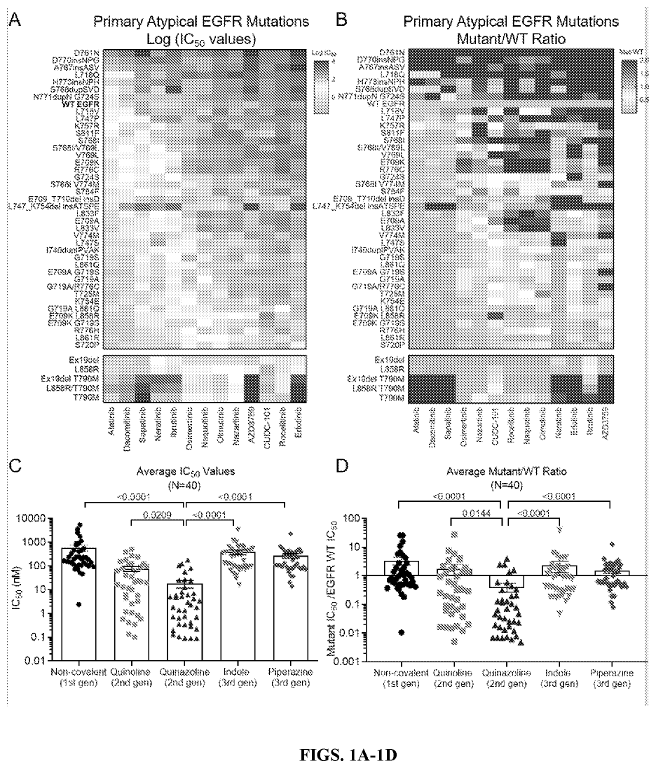

FIGS. 2A-2D: P-loop exon 18 mutations cause primary resistance to osimertinib, but not other EGFR TKIs in vivo. (FIG. 2A) Tumor growth curve of PDX model of NSCLC harboring an EGFR exon 18 P-loop mutation (G719A) treated with the indicated inhibitors for 28 days. (FIG. 2B) Bar graphs of the mean±SEM of percent change in G719A tumor volume at the end of the 28 day experiment after treatment with indicated inhibitors. Symbols are representative of individual mice. Significant differences were determined by ANOVA and Tukey test for multiple comparisons. (FIG. 2C) Tumor growth curve of NSCLC PDX model with non-P-loop exon 18 EGFR mutation (E709K L858R) treated with the indicated inhibitors for 28 days. (FIG. 2D) Bar graphs of the mean±SEM of percent change in E709K/L858R tumor volume at the end of the 28 day experiment after treatment with indicated inhibitors. Symbols are representative of individual mice. Significant differences were determined by ANOVA and Tukey test for multiple comparisons.

FIGS. 3A-3D: Acquired atypical mutations drive resistance to osimertinib, but are sensitive to quinazoline TKIs, and drug sensitivity/resistance profile of co-occurring mutations may be driven by primary mutation. (FIG. 3A) Heatmap of Log IC₅₀ values of Ba/F3 cells expressing acquired atypical mutations spanning exons 18-21 treated with indicated inhibitors for 72 hours. Mutations are ordered from most resistant to most sensitive top to bottom, and drugs are ordered from lowest IC₅₀ value to highest IC₅₀ value left to right. (FIG. 3B) Heatmap of ratio of the IC₅₀ values of Ba/F3 cells expressing acquired atypical mutations spanning exons 18-21 divided by the IC₅₀ values of Ba/F3 cells expressing WT EGFR (+10 ng/ml EGF) treated with indicated inhibitors for 72 hours. Order of mutations was kept the same from panel A, but drugs were re-ordered from left to right for most selective to least selective. (FIGS. 3C-D) Bar Graphs of mutant/WT ratio of Ex19del (FIG. 3C) and L858R (FIG. 3D) primary mutations with and without primary mutations by drug class. For primary mutations alone (open bars) symbols are representative of average mutant/WT ratio for individual drugs, and for primary mutations+atypical mutations (solid bars), symbols are representative of average mutant/WT ratio of all drugs in indicated drug class for each mutation. Statistical differences were determined using a non-parametric students' t-test due to asymmetrical sample size.

FIGS. 4A-4D: T790M mutation drives resistance to first and second generation drugs irrespective of primary or tertiary mutation, but unique third generation TKIs and drug repurposing may overcome tertiary, T790M positive, drug resistance. (FIG. 4A) Heatmap of Log IC₅₀ values of Ba/F3 cells expressing EGFR mutations co-occurring with T790M mutations and treated with indicated classical EGFR inhibitors for 72 hours. Mutations are ordered from most resistant to most sensitive top to bottom, and drugs are ordered from lowest IC₅₀ value to highest IC₅₀ value left to right. (FIG. 4B) Heatmap of ratio of the IC₅₀ values of Ba/F3 cells expressing EGFR mutations co-occurring with T790M mutations divided by the IC₅₀ values of Ba/F3 cells expressing WT EGFR (+10 ng/ml EGF) treated with indicated classical EGFR inhibitors for 72 hours. Order of mutations was kept the same from panel A, but drugs were re-ordered from left to right for most selective to least indicated classical EGFR inhibitors selective. (FIG. 4C) Heatmap of Log IC₅₀ values of Ba/F3 cells expressing EGFR mutations co-occurring with T790M mutations and treated with indicated repurposed inhibitors for 72 hours. Order of mutations was kept the same from panel A, and drugs are ordered from lowest IC₅₀ value to highest IC₅₀ value left to right. (FIG. 4D) Heatmap of ratio of the IC₅₀ values of Ba/F3 cells expressing EGFR mutations co-occurring with T790M mutations divided by the IC₅₀ values of Ba/F3 cells expressing WT EGFR (+10 ng/ml EGF) treated with indicated repurposed inhibitors for 72 hours. Order of mutations was kept the same from panel A, but drugs were re-ordered from left to right for most selective to least selective.

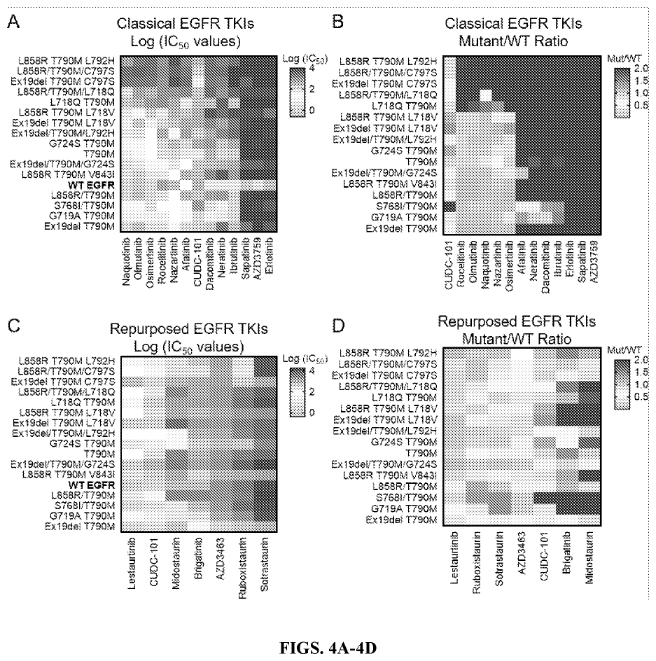

### DESCRIPTION OF ILLUSTRATIVE EMBODIMENTS

The present studies identified osimertinib resistant EGFR mutations across various malignancies, such as NSCLC. Systematically, the drug sensitivity of the resistant mutations across TKIs was evaluated. It was found that the resistant EGFR mutations were sensitive to second generation, quinazolinamine derivative TKIs.

Accordingly, certain embodiments of the present disclosure provide methods for treating cancer patients with osimertinib resistant EGFR mutations. In particular, the present methods comprise the administration of second generation, quinazolinamine derivative TKIs to patients identified to have one or more osimertinib resistant EGFR mutations, such an Exon 18, 19, 20, or 21 mutations, such as acquired atypical mutations and/or classical mutations. The size and flexibility of second generation, quinazolinamine derivative TKIs overcomes steric hindrance, inhibiting EGFR mutants at low nanomolar concentrations. Thus, second generation, quinazolinamine derivative TKIs are potent EGFR inhibitors that can be used to target osimertinib resistant EGFR mutations.

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, “essentially free,” in terms of a specified component, is used herein to mean that none of the specified components has been purposefully formulated into a composition and/or is present only as a contaminant or in trace amounts. The total amount of the specified component resulting from any unintended contamination of a composition is typically below 1%, more preferable below 0.1% and still more preferable below 0.01% of the composition. Most preferred is a composition in which no measurable amount of the specified component can be detected with standard analytical methods.

The term “essentially” is to be understood that methods or compositions include only the specified steps or materials and those that do not materially affect the basic and novel characteristics of those methods and compositions.

The term “substantially free of” is used to 98% of the listed components and less than 2% of the components to which composition or particle is substantially free of.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease. For example, a treatment may include administration of an effective amount of second generation, quinazolinamine derivative TKIs.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “insertion(s)” or “insertion mutation(s)” refers to the addition of one or more nucleotide base pairs into a DNA sequence.

“Hybridize” or “hybridization” refers to the binding between nucleic acids. The conditions for hybridization can be varied according to the sequence homology of the nucleic acids to be bound. Thus, if the sequence homology between the subject nucleic acids is high, stringent conditions are used. If the sequence homology is low, mild conditions are used. When the hybridization conditions are stringent, the hybridization specificity increases, and this increase of the hybridization specificity leads to a decrease in the yield of non-specific hybridization products. However, under mild hybridization conditions, the hybridization specificity decreases, and this decrease in the hybridization specificity leads to an increase in the yield of non-specific hybridization products.

A “probe” or “probes” refers to a polynucleotide that is at least eight (8) nucleotides in length and which forms a hybrid structure with a target sequence, due to complementarity of at least one sequence in the probe with a sequence in the target region. The polynucleotide can be composed of DNA and/or RNA. Probes in certain embodiments, are detectably labeled. Probes can vary significantly in size. Generally, probes are, for example, at least 8 to 15 nucleotides in length. Other probes are, for example, at least 20, 30 or 40 nucleotides long. Still other probes are somewhat longer, being at least, for example, 50, 60, 70, 80, or 90 nucleotides long. Probes can be of any specific length that falls within the foregoing ranges as well. Preferably, the probe does not contain a sequence complementary to the sequence(s) used to prime for a target sequence during the polymerase chain reaction.

“Oligonucleotide” or “polynucleotide” refers to a polymer of a single-stranded or double-stranded deoxyribonucleotide or ribonucleotide, which may be unmodified RNA or DNA or modified RNA or DNA.

A “modified ribonucleotide” or deoxyribonucleotide refer to molecules that can be used in place of naturally occurring bases in nucleic acid and includes, but is not limited to, modified purines and pyrimidines, minor bases, convertible nucleosides, structural analogs of purines and pyrimidines, labeled, derivatized and modified nucleosides and nucleotides, conjugated nucleosides and nucleotides, sequence modifiers, terminus modifiers, spacer modifiers, and nucleotides with backbone modifications, including, but not limited to, ribose-modified nucleotides, phosphoramidates, phosphorothioates, phosphonamidites, methyl phosphonates, methyl phosphoramidites, methyl phosphonamidites, 5′-β-cyanoethyl phosphoramidites, methylenephosphonates, phosphorodithioates, peptide nucleic acids, achiral and neutral internucleotidic linkages.

A “variant” refers to a polynucleotide or polypeptide that differs relative to a wild-type or the most prevalent form in a population of individuals by the exchange, deletion, or insertion of one or more nucleotides or amino acids, respectively. The number of nucleotides or amino acids exchanged, deleted, or inserted can be 1, 2, 3, 4, 5, 6, 7, 8, 9, 10, 11, 12, 13, 14, 15, 16, 17, 18, 19, 20 or more such as 25, 30, 35, 40, 45 or 50.

A “primer” or “primer sequence” refers to an oligonucleotide that hybridizes to a target nucleic acid sequence (for example, a DNA template to be amplified) to prime a nucleic acid synthesis reaction. The primer may be a DNA oligonucleotide, a RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acid under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions.

“Detection,” “detectable” and grammatical equivalents thereof refer to ways of determining the presence and/or quantity and/or identity of a target nucleic acid sequence. In some embodiments, detection occurs amplifying the target nucleic acid sequence. In other embodiments, sequencing of the target nucleic acid can be characterized as “detecting” the target nucleic acid. A label attached to the probe can include any of a variety of different labels known in the art that can be detected by, for example, chemical or physical means. Labels that can be attached to probes may include, for example, fluorescent and luminescence materials.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA) (TwistDx, Cambridge, UK), and self-sustained sequence replication (3 SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3^{rd} Edition).

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

### II. Resistant EGFR Mutations

Certain embodiments of the present disclosure concern determining if a subject has one or more osimertinib resistant EGFR mutations, such an Exon 18, 19, 20, or 21 mutation. The subject may have 2, 3, 4, or more EGFR exon 20 mutations. The one or more EGFR mutations may be located at one or more residues selected from the group consisting of E709, L718, G719, G724, C797, V843, T854, L861, and L792. The one or more EGFR mutations may be G719X, E709X, G724S, L718X, L861Q, T8541, V8431, C797S, and/or L792X, wherein X is any amino acid. Mutation detection methods are known the art including PCR analyses and nucleic acid sequencing as well as FISH and CGH. In particular aspects, the EGFR mutations are detected by DNA sequencing, such as from a tumor or circulating free DNA from plasma.

The EGFR exon 18 mutation(s) may comprise one or more point mutations, insertions, and/or deletions of 3-18 nucleotides between amino acids in-frame deletions of exon 18 between amino acids 688-728. The one or more EGFR exon 18 mutations may be located at one or more residues selected from the group consisting of E709, L718, G719, S5720, and G724. The one or more EGFR exon 18 mutations may comprise E709A, L718Q, L718V, G719A, G719S, S720P, and G724S.

The EGFR exon 19 mutation(s) may comprise one or more point mutations, insertions, and/or deletions of 3-18 nucleotides between amino acids in-frame deletions of exon 19 between amino acids 729-761. The one or more EGFR exon 19 mutations may be located at one or more residues selected from the group consisting of 1744, L747, L747, A755, K757, and/or D761. The one or more EGFR exon 19 mutations may comprise I744V, I744T L747S, L747FS, A755T, K757R, and/or D761N.

The EGFR exon 20 mutation(s) may comprise one or more point mutations, insertions, and/or deletions of 3-18 nucleotides between amino acids 763-823. The one or more EGFR exon 20 mutations may be located at one or more residues selected from the group consisting of G724, A763, S768, V769, H773, V774, C775, S784, G796, C797, S811, and R776. The one or more EGFR exon 20 mutations may comprise S784F, R776C, S7681, V774M, S7681, H773insAH, V774A, V769M, S768dupSVD, A763insLQEA, G796D, S784F, C775Y and/or S811F.

The EGFR exon 21 mutation(s) may comprise one or more point mutations, insertions, and/or deletions of 3-18 nucleotides between amino acids in-frame deletions of exon 21 between amino acids 824-875. The one or more EGFR exon 19 mutations may be located at one or more residues selected from the group consisting of S784, G796, C797, S811, L833, G836, V843, T854, L861, L861, L862, L844 and L858. The one or more EGFR exon 21 mutations may comprise L858R, L833F, L861Q, V843I, L861R, L862V, L844V, L861Q, G836S, and/or T854I.

In some aspects, the subject may have or develop a mutation at EGFR residue C797 which may result in resistance to the TKI, such as second generation, quinazolinamine derivative TKIs. Thus, in certain aspects, the subject is determined to not have a mutation at EGFR C797 and/or T790, such as C797S and/or T790M. In some aspects, subjects with T790 mutations, such as T790M, may be administered osimertinib and subjects with C797 mutations, such as C797S, may be administered chemotherapy and/or radiotherapy. For example, if C797S is acquired in the context of a classical EGFR mutation (e.g., L858R or exon 19 deletion) and T790M is not present, these mutations may be sensitive to quinazolinamine TKIs. However, if a C797S mutation is acquired with a T790M mutation or exon 20 insertion mutation, these mutations may be resistant to quinazolinamine TKIs. Furthermore, if a T790M mutation is acquired with a classical mutation (e.g., L858R or Exon 19 deletion) these mutations may be resistant to quinazolinamine TKIs, but not osimertinib. Also, in vitro, when a T790M mutation is acquired with an exon 18 point mutation, (G719X/T790M), these mutations appear to remain sensitive to quinazolinamine TKIs. In some aspects, L858R/C797S, Ex19del/C797S, or G719X/T790M mutants are sensitive to quinazolinamine TKIs. However, in certain aspects, L858R/T790M/C797S, Exon 19 deletion/T790M/C797s, and exon 20 insertions+C797S or T790M mutants are resistant to EGFR TKIs.

The patient sample can be any bodily tissue or fluid that includes nucleic acids from the lung cancer in the subject. In certain embodiments, the sample will be a blood sample comprising circulating tumor cells or cell free DNA. In other embodiments, the sample can be a tissue, such as a lung tissue. The lung tissue can be from a tumor tissue and may be fresh frozen or formalin-fixed, paraffin-embedded (FFPE). In certain embodiments, a lung tumor FFPE sample is obtained.

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine, tumor, or expectorant. The sample itself will typically include nucleated cells (e.g., blood or buccal cells) or tissue removed from the subject including normal or tumor tissue. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of resistant EGFR mutations as described herein can be determined using methods known in the art. For example, gel electrophoresis, capillary electrophoresis, size exclusion chromatography, sequencing, and/or arrays can be used to detect the presence or absence of insertion mutations. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an insertion mutation as described herein. An insertion mutation can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular variant.

A set of probes typically refers to a set of primers, usually primer pairs, and/or detectably-labeled probes that are used to detect the target genetic variations (e.g., EGFR mutations) used in the actionable treatment recommendations of the present disclosure. The primer pairs are used in an amplification reaction to define an amplicon that spans a region for a target genetic variation for each of the aforementioned genes. The set of amplicons are detected by a set of matched probes. In an exemplary embodiment, the present methods may use TaqMan™ (Roche Molecular Systems, Pleasanton, Calif.) assays that are used to detect a set of target genetic variations, such as EGFR mutations. In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing (Sears et al., 1992), solid-phase sequencing (Zimmerman et al., 1992), sequencing with mass spectrometry such as matrix-assisted laser desorption/ionization time-of-flight mass spectrometry (MALDI-TOF/MS; Fu et al., 1998), and sequencing by hybridization (Chee et al., 1996; Drmanac et al., 1993; Drmanac et al., 1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC, Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

In one example, a method of identifying an EGFR mutation in a sample comprises contacting a nucleic acid from said sample with a nucleic acid probe that is capable of specifically hybridizing to nucleic acid encoding a mutated EGFR protein, or fragment thereof incorporating a mutation, and detecting said hybridization. In a particular embodiment, said probe is detectably labeled such as with a radioisotope (³H, ³²P, or ³³P), a fluorescent agent (rhodamine, or fluorescein) or a chromogenic agent. In a particular embodiment, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, said probes of the present disclosure are provided in a kit for identifying EGFR mutations in a sample, said kit comprising an oligonucleotide that specifically hybridizes to or adjacent to a site of mutation in the EGFR gene. The kit may further comprise instructions for treating patients having tumors that contain EGFR mutations with second generation, quinazolinamine derivative TKIs based on the result of a hybridization test using the kit.

In another aspect, a method for detecting an EGFR mutation in a sample comprises amplifying from said sample nucleic acids corresponding to said EGFR gene, or a fragment thereof suspected of containing a mutation, and comparing the electrophoretic mobility of the amplified nucleic acid to the electrophoretic mobility of corresponding wild-type EGFR gene or fragment thereof. A difference in the mobility indicates the presence of a mutation in the amplified nucleic acid sequence. Electrophoretic mobility may be determined on polyacrylamide gel.

Alternatively, nucleic acids may be analyzed for detection of mutations using Enzymatic Mutation Detection (EMD) (Del Tito et al., 1998). EMD uses the bacteriophage resolvase T4 endonuclease VII, which scans along double-stranded DNA until it detects and cleaves structural distortions caused by base pair mismatches resulting from point mutations, insertions and deletions. Detection of two short fragments formed by resolvase cleavage, for example by gel electrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T4 endonuclease VII as demonstrated in U.S. Pat. No. 5,869,245.

### III. Methods of Treatment

Further provided herein are methods for treating or delaying progression of cancer in an individual comprising administering to the individual an effective amount of second generation, quinazolinamine derivative TKIs, or a structurally similar inhibitor, to a subject determined to have a resistant EGFR mutation. The subject may have more than one EGFR mutations.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is non-small cell lung cancer.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

Certain embodiments concern the administration of second generation, quinazolinamine derivative TKIs to a subject determined to have osimertinib resistant EGFR mutations. The second generation, quinazolinamine derivative TKIs may be administered orally, such as in a tablet. The second generation, quinazolinamine derivative TKIs may be administered in a dose of 4-25 mg, such as at a dose of 5, 6, 7, 8, 9, 10, 11, 12, 13, 14, 15, 16, 17, 18, 19, 20, 21, 22, 23, or 24 mg. The dosing may be daily, every other day, every 3 days or weekly. The dosing may be on a continuous schedule, such as on 28 days cycles.

The osimertinib, chemotherapy, and/or radiation may be administered alone or in combination with second generation, quinazolinamine derivative TKIs. Osimertinib may be administered at a dose of 25 to 100 mg, such as about 40 or 80 mg. The dosing may be daily, every other day, every 2 days, every 3 days, or weekly. The osimertinib may be administered orally, such as in tablet.

A. Pharmaceutical Compositions

Also provided herein are pharmaceutical compositions and formulations comprising second generation, quinazolinamine derivative TKIs and a pharmaceutically acceptable carrier for subjects determined to have a resistant EGFR mutation.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve second generation, quinazolinamine derivative TKIs in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

The second generation, quinazolinamine derivative TKIs may be administered before, during, after, or in various combinations relative to an additional cancer therapy, such as immune checkpoint therapy. The administrations may be in intervals ranging from concurrently to minutes to days to weeks. In embodiments where the second generation, quinazolinamine derivative TKI is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below second generation, quinazolinamine derivative TKI is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine, plicamycin, gemcitabine, navelbine, farnesyl-protein transferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody—drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129; International Patent Publication Nos. WO 01/14424, WO 98/42752, and WO 00/37504 (CP675,206, also known as tremelimumab; formerly ticilimumab); U.S. Pat. No. 6,207,156; Hurwitz et al., 1998; Camacho et al., 2004; and Mokyr et al., 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application Nos. WO2001014424, and WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. Kit

Also within the scope of the present disclosure are kits for detecting osimertinib resistant EGFR mutations, such as those disclosed herein. An example of such a kit may include a set of osimertinib resistant EGFR mutation-specific primers. The kit may further comprise instructions for use of the primers to detect the presence or absence of the specific osimertinib resistant EGFR mutations described herein. The kit may further comprise instructions for diagnostic purposes, indicating that a positive identification of osimertinib resistant EGFR mutations described herein in a sample from a cancer patient indicates sensitivity to the second generation, quinazolinamine derivative TKI or a structurally similar inhibitor. The kit may further comprise instructions that indicate that a positive identification of osimertinib resistant EGFR mutations described herein in a sample from a cancer patient indicates that a patient should be treated with second generation, quinazolinamine derivative TKIs, or a structurally similar inhibitor.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification of Drugs for Cancer Cells with Osimertinib Resistant EGFR Mutations

A panel of Ba/F3 cell lines was generated expressing osimertinib or erlotinib resistant mutations including atypical EGFR mutations spanning exons 18-21 and classical EGFR mutations. The transforming capability of the mutations was then evaluated by sustained cell viability following IL-3 deprivation. Activating EGFR mutant Ba/F3 cells were then screened against EGFR TKIs. Cell viability was determined by the Cell Titer Glo assay.

Second generation, quinazolinamine derivative TKIs inhibited the proliferation of Ba/F3 cell lines expressing atypical mutations such as L861Q, G719S, L858R/L792H, L858R/C797S, and Exl9del/C797S, with IC₅₀ values<3 nM.

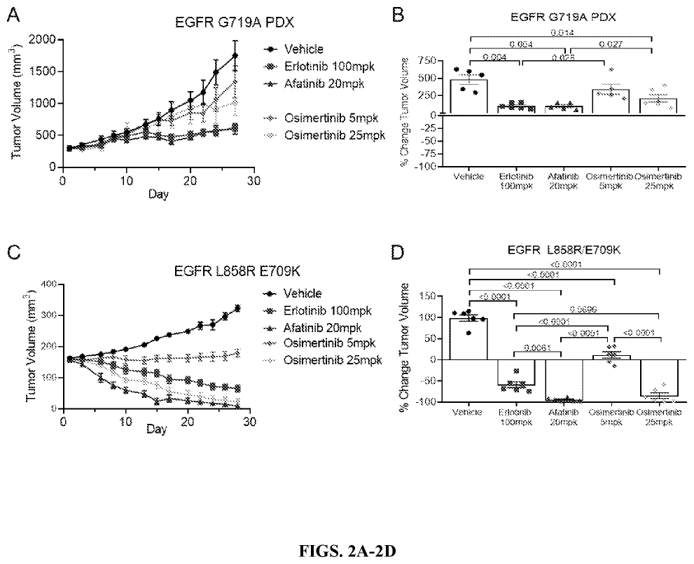

In another study, a PDX model of NSCLC harboring an EGFR exon 18 P-loop mutation (G719A) was treated with the indicated inhibitors for 28 days. It was further found that P-loop exon 18 mutations cause primary resistance to osimertinib, but not other EGFR TKIs in vivo (FIG. 2).

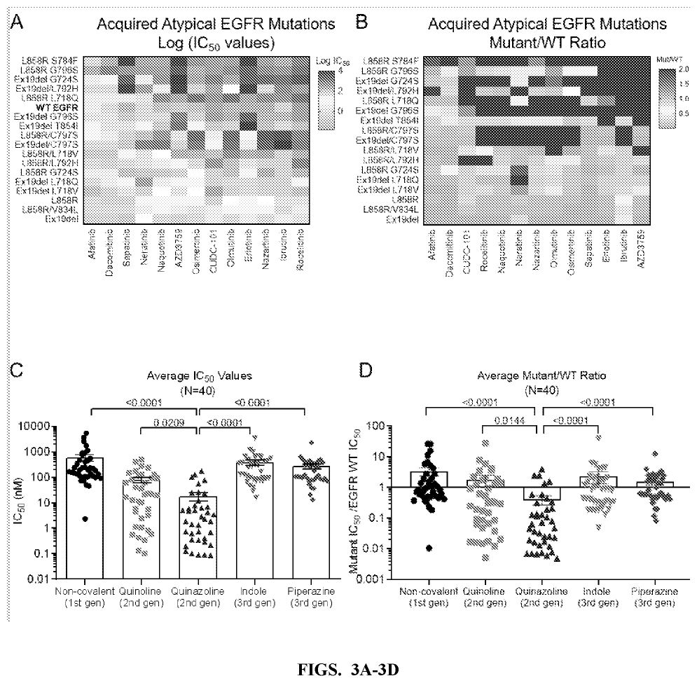

Ba/F3 cells expressing acquired atypical mutations spanning exons 18-21 were treated with inhibitors for 72 hours. It was observed that acquired atypical mutations drive resistance to osimertinib, but were sensitive to quinazoline TKIs, and drug sensitivity/resistance profile of co-occurring mutations may be driven by the primary mutation (FIG. 3). It was also shown that the T790M mutation drives resistance to first and second generation drugs irrespective of the primary or tertiary mutation, but unique third generation TKIs and drug repurposing may overcome tertiary, T790M positive, drug resistance.

TABLE 1
IC50 values of primary atypical EGFR mutations.

| [symbol] alues, nM | Erlotinib | Sapatinib | AZD3759 | CUDC-101 | Afatinib | Dacomitinib | Neratinib |
|---|---|---|---|---|---|---|---|
| [symbol] K | 327.03 | 22.19 | 325.10 | 306.03 | 1.48 | 16.82 | 19.65 |
| E709_T710del | 561.63 | ND | ND | ND | 5.71 | 4.16 | 150.53 |
| insD | | | | | | | |
| E709A | 262.60 | 2.79 | 109.75 | 443.27 | 0.60 | 4.84 | 11.56 |
| E709AG719S | 99.57 | 4.07 | 221.40 | 252.47 | 0.09 | 0.10 | 0.58 |
| E709K L858R | 103.94 | 3.80 | 22.88 | 612.50 | 0.15 | 0.50 | 2.54 |
| E709K G719S | 55.90 | 2.01 | 77.78 | 239.23 | 0.11 | 0.08 | 3.24 |
| L718Q | 1700.67 | 370.60 | 1762.33 | 765.90 | 12.08 | 23.76 | 96.23 |
| L718V | 656.05 | 230.77 | 716.75 | 229.80 | 4.89 | 6.85 | 117.20 |
| G719S | 63.55 | 36.47 | 52.44 | 146.70 | 0.70 | 0.14 | 0.83 |
| G719A | 146.62 | 2.31 | 104.07 | 300.88 | 0.77 | 0.12 | 0.14 |
| G719A/R776C | 143.40 | 8.93 | 208.47 | 192.53 | 0.15 | 0.16 | 0.10 |
| G719A L861Q | 153.81 | 8.42 | 77.49 | 57.65 | 1.30 | 0.14 | 0.34 |
| S720P | 5.57 | 0.79 | 0.75 | ND | 0.07 | 0.49 | 0.58 |
| G724S | 641.84 | 25.73 | 85.98 | 201.80 | 5.32 | 14.76 | 41.78 |
| T725M | 101.83 | 1.22 | 110.02 | 250.67 | 0.18 | 1.90 | 5.73 |
| l740duplPVAK | 94.44 | 1.52 | 93.35 | 237.17 | 1.23 | 0.85 | 6.11 |
| L747P | 472.10 | 5.61 | 305.20 | 553.90 | 4.90 | 18.44 | 248.30 |
| L747 K754del | 5015.67 | 1456.33 | 8117.00 | 140.25 | 37.56 | 339.13 | 399.17 |
| insATSPE | | | | | | | |
| L747S | 114.16 | 0.95 | 84.39 | 202.93 | 0.41 | 0.40 | 277.67 |
| K754E | 110.23 | 0.56 | 48.33 | 206.33 | 0.23 | 2.01 | 4.43 |
| K757R | 260.47 | 59.11 | 381.85 | 200.00 | 20.00 | 50.15 | 23.64 |
| A767insASV | 16773.67 | 407.57 | 4316.00 | 505.70 | 128.91 | 189.67 | 236.60 |
| S768dupSVD | 3162.00 | 486.67 | 3194.67 | 305.87 | 24.82 | 54.73 | 62.57 |
| S768I | 396.10 | 5.72 | 358.47 | 888.20 | 1.86 | 3.50 | 63.23 |
| S768I/V769L | 907.03 | 47.38 | 734.77 | 203.60 | 4.38 | 12.94 | 13.14 |
| S768I V774M | 267.60 | 48.33 | 265.50 | 200.00 | 4.95 | 8.73 | 81.97 |
| V769L | 1293.00 | 36.36 | 1014.53 | 792.37 | 6.01 | 8.70 | 10.13 |
| D770insNPG | 4646.33 | 916.67 | 3985.00 | 924.87 | 198.20 | 335.37 | 1873.47 |
| N771dupN | 1701.67 | 134.64 | 1307.07 | 509.00 | 42.36 | 196.18 | 129.15 |
| G724S | | | | | | | |
| H773insNPH | 1545.00 | 230.83 | 1622.33 | 352.10 | 64.88 | 102.08 | 518.90 |
| [symbol] M | 324.33 | 10.18 | 326.17 | 286.23 | 1.09 | 2.80 | 127.09 |
| [symbol] C | 367.87 | 3.67 | 158.11 | 446.03 | 1.20 | 7.54 | 16.88 |
| [symbol] H | 50.67 | 0.35 | 22.70 | 110.09 | 0.10 | 0.08 | 1.08 |
| [symbol] F | 136.83 | 15.53 | 72.93 | 245.43 | 1.76 | 4.83 | 35.63 |
| S811F | 332.30 | 23.09 | 145.00 | ND | 7.43 | 67.09 | 90.98 |
| L833F | 171.72 | 1.21 | 129.74 | 331.80 | 1.06 | 11.56 | 21.86 |
| L833V | 240.85 | 2.30 | 138.70 | 214.35 | 0.28 | 3.91 | 21.23 |
| L861Q | 173.63 | 2.31 | 92.72 | 177.50 | 0.97 | 0.13 | 1.16 |
| L861R | 95.91 | 0.51 | 38.49 | 71.36 | 0.13 | 0.10 | 0.42 |

| [symbol] alues, nM | Ibrutinib | Osimertinib | Rociletinib | Nazartinib | Olmutinib | Naquotini [symbol] |
|---|---|---|---|---|---|---|
| [symbol] K | 30.99 | 255.17 | 1403.00 | 388.40 | 655.97 | 358. [symbol] |
| E709_T710del | ND | 171.41 | ND | ND | 271.60 | [symbol] |
| insD | | | | | | |
| E709A | 32.83 | 56.22 | 1157.25 | 174.61 | 372.45 | 254. [symbol] |
| E709AG719S | 2.02 | 69.01 | 207.53 | 119.77 | 144.47 | 48. [symbol] |
| E709K L858R | 21.11 | 7.83 | 61.66 | 25.83 | 16.12 | 11.02 |
| E709K G719S | 0.33 | 54.22 | 94.44 | 43.97 | 57.34 | 15.23 |
| L718Q | 521.63 | 1532.00 | 1142.00 | 1614.00 | 3982.67 | 733.60 |
| L718V | 168.35 | 171.70 | 1367.90 | 1021.55 | 139.17 | 78.41 |
| G719S | 17.09 | 130.21 | 150.32 | 105.87 | 214.26 | 36.72 |
| G719A | 10.76 | 105.12 | 145.47 | 87.85 | 141.19 | 18.11 |
| G719A/R776C | 4.42 | 63.41 | 230.40 | 56.55 | 180.73 | 39.03 |
| G719A L861Q | 27.10 | 31.01 | 36.07 | 68.93 | 73.98 | 4.22 |
| S720P | ND | 24.87 | 160.10 | 22.63 | 87.50 | 52.95 |
| G724S | 19.16 | 67.28 | 306.63 | 126.76 | 82.95 | 58.07 |
| T725M | 5.23 | 47.20 | 365.47 | 58.88 | 280.02 | 77.58 |
| l740duplPVAK | 10.68 | 28.10 | 433.23 | 75.36 | 138.36 | 67.67 |
| L747P | 424.40 | 93.79 | 630.23 | 287.97 | 392.93 | 472.23 |
| L747 K754del | 1741.00 | 0.40 | 0.92 | 1.55 | 0.14 | 0.58 |
| insATSPE | | | | | | |
| L747S | 34.59 | 25.91 | 247.70 | 81.72 | 62.99 | 120.00 |
| K754E | 2.62 | 12.67 | 217.87 | 61.57 | 88.50 | 66.00 |
| K757R | 82.34 | 120.00 | 709.45 | 623.85 | 144.07 | 122.38 |
| A767insASV | 513.40 | 342.67 | 2479.67 | 827.50 | 680.17 | 216. [symbol] |
| S768dupSVD | 11865.67 | 143.53 | 1152.40 | 519.73 | 554.07 | 152. [symbol] |
| S768I | 159.30 | 210.47 | 1738.00 | 513.60 | 837.40 | 503. [symbol] |
| S768I/V769L | 68.70 | 90.94 | 570.73 | 292.17 | 358.70 | 233. [symbol] |
| S768I V774M | 71.19 | 3.26 | 160.43 | 124.00 | 111.10 | 66. [symbol] |
| V769L | 5.70 | 165.27 | 1040.60 | 253.80 | 312.57 | 218. [symbol] |
| D770insNPG | 983.27 | 191.60 | 914.83 | 685.97 | 310.47 | 174.57 |
| N771dupN | 123.80 | 170.34 | 1143.00 | 438.73 | 417.07 | 374.50 |
| G724S | | | | | | |
| H773insNPH | 955.10 | 133.83 | 1165.33 | 550.10 | 428.00 | 145.05 |
| [symbol] M | 0.53 | 4.28 | 109.43 | 152.93 | 69.97 | 38.54 |
| [symbol] C | 74.87 | 235.13 | 1860.00 | 369.03 | 513.77 | 575.00 |
| [symbol] H | 1.13 | 17.82 | 248.30 | 46.69 | 114.73 | 68. [symbol] |
| [symbol] F | 21.35 | 101.99 | 690.27 | 402.90 | 140.17 | 70. [symbol] |
| S811F | ND | 96.17 | 845.50 | 742.80 | 760.60 | 173. [symbol] |
| L833F | 27.84 | 101.38 | 806.83 | 252.89 | 329.03 | 411. [symbol] |
| L833V | 28.10 | 63.44 | 803.30 | 246.20 | 394.40 | 402. [symbol] |
| L861Q | 29.63 | 27.27 | 201.00 | 83.40 | 83.88 | 77.43 |
| L861R | 1.41 | 6.55 | 42.80 | 14.53 | 29.41 | 12.19 |

[symbol]

indicates data missing or illegible when filed

TABLE 2
Acquired osimertinib resistance mutations (T790M negative).

| IC₅₀ Values, nM | Erlotinib | Afatinib | Dacomitinib | Neratinib | Osimertinib | Nazartinib | Olmutinib |
|---|---|---|---|---|---|---|---|
| L858R S784F | 10000.00 | 56.49 | 226.27 | 298.87 | 580.67 | 427.93 | 437.33 |
| Ex19del G724S | 3134.50 | 3.16 | 14.53 | 30.97 | 316.33 | 3021.33 | 331.37 |
| Ex19del/L792H | 9745.67 | 62.74 | 51.78 | 453.53 | 87.00 | 56.70 | 19.26 |
| Ex19del/C797S | 2.09 | 0.90 | 1.09 | 1052.93 | 1442.27 | 3977.00 | 2143.00 |
| Ex19delT854l | 17937.50 | 5.69 | 44.74 | 27.58 | 24.12 | 138.07 | 95.02 |
| L858R/C797S | 7.93 | 4.74 | 2.82 | 156.10 | 2369.00 | 4487.00 | 2245.00 |
| L858R G796S | 3742.00 | 22.27 | 22.67 | 430.43 | 296.77 | 856.33 | 957.90 |
| L858R L718Q | 400.90 | 2.60 | 3.17 | 42.74 | 1811.60 | 808.40 | 2327.33 |
| Ex19del G796S | 3962.00 | 34.88 | 44.72 | 49.64 | 70.34 | 210.18 | 254.27 |
| L858R/L792H | 39.97 | 0.53 | 0.18 | 3.11 | 59.12 | 74.41 | 140.69 |
| L858R/L718V | 219.67 | 0.09 | 0.09 | 1.50 | 168.69 | 335.07 | 864.03 |
| L858R G724S | 4.95 | 4.09 | 32.28 | 116.40 | 19.12 | 248.20 | 164.80 |
| Ex19del L718Q | 50.24 | 0.69 | 4.13 | 499.00 | 22.63 | 72.01 | 52.75 |
| Ex19del L718V | 64.96 | 0.05 | 0.38 | 87.17 | 13.92 | 86.60 | 87.68 |
| L858R/V834L | 7.74 | 0.35 | 0.58 | 2.66 | 1.02 | 14.56 | 6.73 |

| | | | | | CUDC- | |
|---|---|---|---|---|---|---|
| IC₅₀ Values, nM | Rociletinib | Naquotinib | Sapatinib | AZD3759 | 101 | Ibrutinib |
| L858R S784F | 1083.87 | 353.73 | 10000.00 | 10000.00 | 452.23 | 232.90 |
| Ex19del G724S | 5805.50 | 697.45 | 3850.00 | 17022.00 | 974.05 | 1865.00 |
| Ex19del/L792H | 504.17 | 3.94 | 9760.33 | 10000.00 | 719.80 | 745.63 |
| Ex19del/C797S | 1448.67 | 1541.33 | 0.11 | 0.14 | 9.19 | 11008.00 |
| Ex19delT854l | 149.85 | 112.39 | 46.51 | 1675.50 | 108.10 | 66.18 |
| L858R/C797S | 1166.80 | 709.13 | 0.20 | 1.97 | 34.40 | 6882.00 |
| L858R G796S | 1704.67 | 423.03 | 624.90 | 4243.33 | 451.53 | 535.53 |
| L858R L718Q | 3961.03 | 747.00 | 35.80 | 391.23 | 698.43 | 649.50 |
| Ex19del G796S | 453.25 | 53.17 | 120.05 | 1249.24 | 419.40 | 169.60 |
| L858R/L792H | 1263.37 | 34.33 | 2.26 | 32.47 | 1030.93 | ND |
| L858R/L718V | 274.20 | 18.83 | 10.18 | 250.20 | 134.73 | 71.73 |
| L858R G724S | 177.00 | 108.40 | 0.04 | 0.28 | ND | ND |
| Ex19del L718Q | 23.19 | 2.64 | 9.97 | 21.60 | ND | ND |
| Ex19del L718V | 131.70 | 13.94 | 1.12 | 42.88 | 300.85 | 67.60 |
| L858R/V834L | 33.59 | 7.19 | 0.34 | 2.33 | 37.90 | 64.39 |

TABLE 3
Acquired Resistance mutations (T790M positive) - Traditional EGFR TKIs.

| | | | | | | | CUDC- |
|---|---|---|---|---|---|---|---|
| [symbol] alues, nM | Naquotinib | Olmutinib | Osimertinib | Rociletinib | Nazartinib | Afatinib | 101 |
| [symbol] /T790M L792H | 624.07 | 2220.67 | 2503.00 | 1005.87 | 5565.67 | 1411.50 | 342.70 |
| [symbol] /T790M/C797S | 4349.00 | 4116.33 | 2931.00 | 1421.67 | 3434.67 | 1937.33 | 274.08 |
| [symbol] del T790M C797S | 1971.67 | 1928.26 | 2328.68 | 1106.66 | 7205.56 | 2504.81 | 58.96 |
| L858R/T790M/L718Q | 91.78 | 620.43 | 332.70 | 1112.20 | 1000.20 | 1344.33 | 217.67 |
| L718Q T790M | 212.30 | 358.37 | 829.13 | 1029.30 | 411.00 | 69.50 | 275.57 |
| L858R T790M L718V | 4.03 | 21.68 | 189.70 | 70.13 | 336.33 | 218.10 | 467.25 |
| Ex19delT790M L718V | 7.72 | 189.57 | 53.09 | 108.52 | 211.37 | 458.73 | 393.90 |
| Ex19del/T790M/L792H | 4.75 | 4.76 | 38.57 | 497.10 | 37.40 | 669.27 | 173.33 |
| G724S T790M | 18.78 | 19.88 | 37.06 | 120.32 | 242.11 | 112.63 | 413.53 |
| T790M | 54.37 | 46.17 | 34.88 | 103.19 | 151.23 | 85.06 | 77.97 |
| Ex19del/T790M/G724S | 3.84 | 5.08 | 21.04 | 39.66 | 79.32 | 32.94 | 245.83 |
| L858R T790M V843I | 2.05 | 0.77 | 6.08 | 4.74 | 32.39 | 331.67 | 273.43 |
| L858R/T790M | 1.88 | 0.79 | 1.84 | 4.21 | 25.38 | 335.27 | 240.43 |
| S768I/T790M | 19.20 | 21.53 | 3.11 | 25.89 | 25.55 | 23.87 | 814.70 |
| G719A T790M | 11.72 | 11.41 | 49.78 | 13.01 | 34.40 | 41.61 | 136.47 |
| Ex19delT790M | 1.41 | 0.41 | 2.80 | 4.77 | 16.40 | 127.51 | 151.52 |

| [symbol] alues, nM | Dacomitinib | Neratinib | Ibrutinib | Sapatinib | AZD3759 | Erlotinib |
|---|---|---|---|---|---|---|
| [symbol] /T790M L792H | 3010.00 | 1635.37 | 2654.33 | 82444.98 | >10000 | >10000 |
| [symbol] /T790M/C797S | 2587.00 | 2815.67 | >10000 | >10000 | >10000 | >10000 |
| [symbol] del T790M C797S | 2360.05 | 1782.95 | 9293.04 | 3126.67 | >10000 | >10000 |
| L858R/T790M/L718Q | 2840.67 | 1069.67 | 3676.33 | 5000.00 | >5000 | >5000 |
| L718Q T790M | 296.87 | 3560.00 | 217.40 | >10000 | >10000 | >10000 |
| L858R T790M L718V | 5561.00 | 2714.67 | >5000 | >5000 | >5000 | 3490.85 |
| Ex19delT790M L718V | 1281.67 | 591.33 | 2023.00 | 7572.33 | >10000 | 74558.35 |
| Ex19del/T790M/L792H | 514.50 | 1338.13 | 2972.33 | >10000 | >10000 | 7093.00 |
| G724S T790M | 747.83 | 217.83 | 406.20 | 8874.67 | 8000.67 | 8427.33 |
| T790M | 376.09 | 121.51 | 148.42 | 12869.00 | >10000 | >10000 |
| Ex19del/T790M/G724S | 243.65 | 354.45 | 501.40 | 4773.67 | 4826.00 | 5294.00 |
| L858R T790M V843I | 1244.67 | 4677.66 | 780.20 | 8943.67 | >10000 | >10000 |
| L858R/T790M | 620.77 | 831.13 | 163.90 | 7093.33 | >10000 | >10000 |
| S768I/T790M | 79.55 | 47.17 | 109.04 | >10000 | 7500.00 | 4943.34 |
| G719A T790M | 57.06 | 65.10 | 118.70 | >10000 | >10000 | 8879.51 |
| Ex19delT790M | 302.23 | 2295.38 | 251.10 | 1959.00 | 13176.66 | 4973.66 |

[symbol]

indicates data missing or illegible when filed

TABLE 4
Acquired Resistance mutations (T790M positive) - Untraditional/Repurposed EGFR TKIs.

| IC50 values, nM | Lestaurtinib | Midostaurin | Brigatinib | AZD3463 | Ruboxistaurin | Sotrastaurin |
|---|---|---|---|---|---|---|
| L858R T790M L792H | 116.60 | 270.67 | 659.93 | 1546.00 | 331.70 | 10728.00 |
| L858R/T790M/C797S | 102.13 | 251.00 | 290.83 | 1271.87 | 364.48 | 9867.33 |
| Ex19del T790M C797S | 0.53 | 880.53 | 1019.57 | 1444.00 | 486.70 | 1041.00 |
| L858R/T790M/L718Q | 78.22 | 2976.00 | 1073.27 | 1120.67 | 3868.33 | 6936.33 |
| L718Q T790M | 115.87 | 1568.00 | 1489.00 | 1141.00 | 4360.00 | 9785.33 |
| L858R T790M L718V | 83.95 | 1378.60 | 772.50 | 662.27 | 2045.00 | 4240.50 |
| Ex19del T790M L718V | 287.03 | 3545.33 | 1095.67 | 1342.33 | 2289.00 | 3333.67 |
| Ex19del/T790M/L792H | 97.10 | 119.43 | 979.03 | 1094.00 | 2520.33 | 5809.00 |
| G724S T790M | 12.80 | 143.67 | 454.13 | 695.00 | 546.93 | 2887.67 |
| T790M | 93.78 | 2945.67 | 858.93 | 1269.00 | 3911.67 | 5790.00 |
| [symbol] 9del/T790M/G724S | 364.25 | 3607.67 | 1537.00 | 1611.33 | 6571.00 | 20067.00 |
| [symbol] 8R T790M V843I | 15.43 | 1328.30 | 350.83 | 768.27 | 1002.47 | 1424.47 |
| [symbol] EGFR | 416.90 | 755.33 | 684.23 | 1632.33 | 4879.00 | 13959.33 |
| [symbol] 8R/T790M | 216.70 | 2861.33 | 2613.00 | 2404.33 | 3842.67 | 19878.33 |
| S768I/T790M | 297.05 | 194.73 | 770.47 | 1732.67 | 5923.33 | 14076.33 |
| G719A T790M | 141.20 | 213.80 | 336.37 | 1340.70 | 2487.67 | 10471.01 |
| Ex19del T790M | 3.71 | 526.30 | 140.60 | 255.10 | 633.10 | 863.07 |

[symbol]

indicates data missing or illegible when filed

TABLE 5
EGFR mutant Vectors.

| | Starting | | |
|---|---|---|---|
| EGFR Mutation | Vector | Genomic change | Manufacturer |
| A767insASV | N/A | N/A | Addgene #32066 |
| D770insNPG | N/A | N/A | Addgene #11016 |
| Ex19del (746_A750del) | N/A | N/A | Addgene #32062 |
| Ex19del/T790M | N/A | N/A | Addgene #32072 |
| G719S | N/A | N/A | Addgene #11013 |
| L858R | N/A | N/A | Addgene #11012 |
| L858R/T790M | N/A | N/A | Addgene #32073 |
| L861Q | N/A | N/A | Addgene #32068 |
| T790M | N/A | N/A | Addgene #32070 |
| WT | N/A | N/A | Addgene #11011 |
| D761N | EGFR WT | c.2281G > A | GeneScript |
| E709_T710del insD | EGFR WT | c.2127_2129del | GeneScript |
| E709A | EGFR WT | c.2126A > C | GeneScript |
| E709A G719S | EGFR G719S | c.2126A > C | GeneScript |
| E709K | EGFR WT | c.2125G > A | GeneScript |
| E709K G719S | EGFR G719S | c.2125G > A | GeneScript |
| E709K L858R | EGFR L858R | c.2125G > A | GeneScript |
| Ex19del G724S | EGFR | c.2313_2314insAAC | GeneScript |
| | Ex19del | | |
| Ex19del G796S | EGFR | c.2386G > A | GeneScript |
| | Ex19del | | |
| Ex19del L718Q | EGFR | c.2153T > A | Bioinnovatise |
| | Ex19del | | |
| Ex19del L718V | EGFR | c.2152C > G | Bioinnovatise |
| | Ex19del | | |
| Ex19del T854I | EGFR | c.2561C > T | GeneScript |
| | Ex19del | | |
| Ex19del/C797S | EGFR | c.2386, T > A | Bioinnovatise |
| | Ex19del | | |
| Ex19del/L792H | EGFR | c.2375T > A | Bioinnovatise |
| | Ex19del | | |
| G719A | EGFR WT | c.2156G > C | Bioinnovatise |
| G719A L861Q | EGFR G719A | c.2582T > A | Bioinnovatise |
| G719A/R776C | EGFR G719A | c.2326C > T | GeneScript |
| G724S | EGFR WT | c.2170G > A | GeneScript |
| H773insNPH | EGFR WT | c.2319_2320insAACCCCCAC | Bioinnovatise |
| I740duplPVAK | EGFR WT | c.2214_2231dup | Bioinnovatise |
| K754E | EGFR WT | c.2260A > G | GeneScript |
| K757R | EGFR WT | c.2270A > G | GeneScript |
| L718Q | EGFR WT | c.2153T > A | GeneScript |
| L718V | EGFR WT | c.2152C > G | GeneScript |
| L747_K754del | EGFR WT | c.2239_2260delinsGCAACATCTCCGG | GeneScript |
| insATSPE | | (SEQ ID NO: 1) | |
| L747P | EGFR WT | c.2239_2240TT > CC | GeneScript |
| L747S | EGFR WT | c.2240T > C | GeneScript |
| L833F | EGFR WT | c.2499G > T | GeneScript |
| L833V | EGFR WT | c.2497T > G | GeneScript |
| L858R G724S | EGFR L858R | c.2313_2314insAAC | GeneScript |
| L858R G796S | EGFR L858R | c.2386G > A | GeneScript |
| L858R L718Q | EGFR L858R | c.2153T > A | GeneScript |
| L858R S784F | EGFR L858R | c.2351C > T | GeneScript |
| L858R/C797S | EGFR L858R | c.2386, T > A | Bioinnovatise |
| L858R/L718V | EGFR L858R | c.2152C > G | GeneScript |
| L858R/L792H | EGFR L858R | c.2375T > A | Bioinnovatise |
| L858R/V834L | EGFR L858R | c.2500G > T | Bioinnovatise |
| L861R | EGFR WT | c.2582T > G | GeneScript |
| N771dupN G724S | EGFR G724S | c.2313_2314insAAC | GeneScript |
| R776C | EGFR WT | c.2326C > T | GeneScript |
| R776H | EGFR WT | c.2327G > A | GeneScript |
| S720P | EGFR WT | c.2158T > C | GeneScript |
| S768dupSVD | EGFR WT | c.2303_2304dupAGCGTGGAC | Bioinnovatise |
| S768I | EGFR WT | c.2303G > T | Bioinnovatise |
| S768I V774M | EGFR S768I | c.2320G > A | GeneScript |
| S768I/T790M | EGFR S768I | c.2369C > T | Bioinnovatise |
| S768I/V769L | EGFR S768I | c.2305G > T | GeneScript |
| S784F | EGFR WT | c.2351C > T | GeneScript |
| S811F | EGFR WT | c.2432C > T | GeneScript |
| T725M | EGFR WT | c.2174C > T | GeneScript |
| V769L | EGFR WT | c.2305G > T | Bioinnovatise |
| V774M | EGFR WT | c.2320G > A | GeneScript |

Thus, second generation, quinazolinamine derivative TKIs are effective inhibitors for both de novo and acquired atypical, osimertinib resistant EGFR mutant NSCLC including L861Q, G719S, L858R/L792H, L858R/C797S, and Exl9del/C797S. The present studies showed that second generation TKIs overcame osimertinib resistance in atypical EGFR mutant NSCLC.

### Example 2—Materials and Methods

Ba/F3 Cell line generation and IL-3 deprivation: Ba/F3 cell lines were established as previously described (Robichaux et al., 2018). Briefly, stable Ba/F3 cell lines were generated by retroviral transduction of Ba/F3 cell line for 12 hours. Retroviruses were generated by transfecting pBabe-Puro based vectors summarized in Table 1 (Addgene and Bioinnovatise) into Phoenix 293T-ampho cells (Orbigen) using Lipofectamine 2000 (Invitrogen). Three days after transduction, 2 μg/ml puromycin (Invitrogen) was added to the RPMI media. Cell lines were then grown in the absence of IL-3 for two weeks and cell viability was assessed every three days using the Cell Titer Glo assay (Progema). Resulting stable cell lines were maintained in RPMI-1640 media containing 10% FBS without IL-3.

Cell Viability Assay and IC₅₀ Estimation: Cell viability was determined using the Cell Titer Glo assay (Promega) as previously described (Robichaux et al., 2018). Briefly, 2000-3000 cells per well were plated in 384-well plates (Greiner Bio-One) in technical triplicate. Cells were treated with seven different concentrations of tyrosine kinase inhibitors or vehicle alone at a final volume of 404, per well. After 3 days, 11 μL of Cell Titer Glo was added to each well. Plates were shaken for 15 minutes, and bioluminescence was determined using a FLUOstar OPTIMA multi-mode micro-plate reader (BMG LABTECH). Bioluminescence values were normalized to DMSO treated cells, and normalized values were plotted in GraphPad Prism using non-linear regression fit to normalized data with a variable slope. 1050 values were calculated by GraphPad Prism at 50% inhibition.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arcila et al., Clin Cancer Res 18:4910-8, 2012.
Arcila et al., Mol Cancer Ther 12(2):220-229, 2013.
Austin-Ward and Villaseca, Revista Medica de Chile, 126(7):838-845, 1998.
Ausubel et al., Current Protocols in Molecular Biology, John Wiley & amp; Sons, New York, N.Y., 2003.
Bukowski et al., Clinical Cancer Res., 4(10):2337-2347, 1998.
Camacho et al. J Clin Oncology 22(145): Abstract No. 2505 (antibody CP-675206), 2004.
Cha et al. Int J Cancer 130:2445-54, 2012.
Chee et al., Science, 274:610-614, 1996.
Cho et al., Cancer Res 73:6770-9, 2013.
Christodoulides et al., Microbiology, 144(Pt 11):3027-3037, 1998.
Church and Gilbert, Proc. Natl. Acad. Sci. USA 81:1991-1995 (1988).
Cotton et al., Proc. Natl. Acad. Sci. USA 85:4397-4401 (1985).
Davidson et al., J Immunother 21(5):389-398, 1998.
Davies et al., Plos One 8, 2013.
Del Tito et al., Clinical Chemistry 44:731-739, 1998.
Drmanac et al., Nat. Biotechnol., 16:54-58, 1998.
Drmanac et al., Science, 260:1649-1652, 1993.
Ettinger et al., J Natl Compr Canc Netw 16:807-21, 2018.
Flavell et al., Cell 15:25 (1978).
Fu et al., Nat. Biotechnol., 16:381-384, 1998/
Geever et al., Proc. Natl. Acad. Sci. USA 78:5081 (1981).
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hong et al., J Biol Chem 282:19781-7, 2007.
Hui and Hashimoto, Infection Immun., 66(11):5329-5336, 1998.
Hurwitz et al. Proc Natl Acad Sci USA 95(17): 10067-10071, 1998.
Hyman et al., Nature 554:189-94, 2018.
International Patent Publication No. WO 99/57318
International Patent Publication No. WO1995001994
International Patent Publication No. WO1998042752
International Patent Publication No. WO2000037504
International Patent Publication No. WO2001014424
International Patent Publication No. WO2009/101611
International Patent Publication No. WO2009/114335
International Patent Publication No. WO2010/027827
International Patent Publication No. WO2011/066342
International Patent Publication No. WO2015016718
International Patent Publication No. WO 00/37504
International Patent Publication No. WO01/14424
International Patent Publication No. WO98/42752
Kosaka et al., Cancer Res 2017.
Kris et al., Ann Oncol 26:1421-7, 2015.
Kris et al., Ann Oncol 26:1421-7, 2015.
Leal, M., Ann N Y Acad Sci 1321, 41-54, 2014.
Lynch et al., N Engl J Med. 350(21):2129-2139, 2004.
Ma et al., J Clin Oncol 33, 2015.
Maemondo et al., N Engl J Med 362:2380-8, 2010.
Meric-Bernstam et al., Clin Cancer Res, 2018.
Mitsudomi and Yatabe, Cancer Sci. 98(12):1817-1824, 2007.
Mokyr et al. Cancer Res 58:5301-5304, 1998.
Oxnard et al., J Thorac Oncol. 8(2):179-184, 2013.
Paez et al., Science 304(5676):1497-1500, 2004.
Pao et al., Proc Natl Acad Sci USA 101(36):13306-13311, 2004.
Pardoll, Nat Rev Cancer, 12(4): 252-64, 2012.
Perera et al., Proc Natl Acad Sci USA 106:474-9, 2009.
Qin et al., Proc. Natl. Acad. Sci. USA, 95(24):14411-14416, 1998.
Raca et al., Genet Test 8(4):387-94 (2004).
Robichaux et al., Nat Med 24:638-46, 2018.
Sanger et al., Proc. Natl. Acad. Sci. USA 74:5463-5467 (1977).
Sears et al., Biotechniques, 13:626-633, 1992.
Sheffield et al., Proc. Natl. Acad. Sci. USA 86:232-236 (1989).
Shen et al., J Recept Signal Transduct Res 36:89-97, 2016.
Thress et al., Nat Med 21:560-2, 2015.
U.S. Pat. No. 4,870,287
U.S. Pat. No. 5,288,644
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,869,245
U.S. Pat. No. 5,885,796
U.S. Pat. No. 6,207,156
U.S. Pat. No. 8,008,449
U.S. Pat. No. 8,017,114
U.S. Pat. No. 8,119,129
U.S. Pat. No. 8,188,102
U.S. Pat. No. 8,329,867
U.S. Pat. No. 8,354,509
U.S. Pat. No. 8,735,553
U.S. Patent Publication No. 2004/0014095
U.S. Patent Publication No. 2005/0260186
U.S. Patent Publication No. 2006/0104968
U.S. Patent Publication No. 20110008369
U.S. Patent Publication No. 20130071452
U.S. Patent Publication No. 2014022021
U.S. Patent Publication No. 20140294898
Underhill et al., Genome Res. 7:996-1005 (1997).
Vogel et al., J Clin Oncol 20:719-26, 2002.
Yang et al., Int J Cancer 2016.
Yasuda et al., Sci Transl Med 5(216):216ra177, 2013.
Zimmerman et al., Methods Mol. Cell. Biol., 3:39-42, 1992.

What is claimed is:

1. A method of treating cancer in a subject comprising administering an effective amount of a quinazolinamine derivative tyrosine kinase inhibitor (TKI) to the subject, wherein the subject has been determined to have one or more epidermal growth factor receptor (EGFR) TKI resistant mutations.

2. The method of claim 1, wherein the EGFR TKI resistant mutations are acquired atypical EGFR mutations.

3. The method of any of claims 1-2, wherein the quinazolinamine derivative TKI is a covalently binding quinazolinamine TKI.

4. The method of claim 3, wherein the covalently binding quinazolinamine TKI is afatinib, tarloxotinib-TKI, dacomitinib, pelitinib, or allitinib.

5. The method of any of claims 1-2, wherein the quinazolinamine derivative TKI is a non-covalently binding quinazolinamine TKI.

6. The method of claim 5, wherein the non-covalently binding quinazolinamine TKI is sapatinib, AZD3759, Varlitinib, TAK-285, or gefitinib.

7. The method of any of claims 1-6, wherein the quinazolinamine derivative TKI is formulated as a tablet.

8. The method of any of claims 1-7, wherein the one or more EGFR TKI resistant mutations comprise a point mutation, insertion, and/or deletion of 1-18 nucleotides at exon 18, 19, 20, or 21.

9. The method of any of claims 1-8, wherein the one or more EGFR TKI resistant mutations comprise one or more point mutations, insertions, and/or deletions of 3-18 nucleotides between amino acids 688-728 of exon 18.

10. The method of claim 9, wherein the one or more EGFR exon 18 mutations are located at one or more residues selected from the group consisting of E709, L718, G719, S720, and G724.

11. The method of claim 9 or 10, wherein the one or more EGFR exon 18 mutations comprise E709A, L718Q, L718V, G719A, G719S, S720P, and/or G724S.

12. The method of any of claims 8-11, wherein the one or more EGFR exon 18 mutations comprise E709A, E790K, L718Q, L718V, G719A, G719S, S720P, and/or G724S.

13. The method of any of claims 1-12, wherein the one or more EGFR TKI resistant mutations comprise one or more point mutations, insertions, and/or deletions of 3-18 nucleotides between amino acids 729-761 of exon 19.

14. The method of claim 13, wherein the one or more EGFR exon 19 mutations are located at one or more residues selected from the group consisting of 1744, L747, L747, A755, K757, and/or D761.

15. The method of claim 13 or 14, wherein the one or more EGFR exon 19 mutations comprise I744V, I744T L747S, L747FS, A755T, K757R, and/or D761N.

16. The method of any of claims 1-15, wherein the one or more EGFR TKI resistant mutations comprise one or more point mutations, insertions, and/or deletions of 3-18 nucleotides between amino acids 763-823 of exon 20.

17. The method of claim 16, wherein the one or more EGFR exon 20 mutations are located at one or more residues selected from the group consisting of A763, S768, V769, H773, D770, V774, C775, S784, L792, G796, C797, S811, and R776.

18. The method of claim 16 or 17, wherein the one or more EGFR exon 20 mutations comprise D770insNPG, S784F, R776C, S7681, V774M, S7681, H773insAH, H773insNPH, V774A, V769L, V769M, S768dupSVD, A763insLQEA, L792H, G796D, S784F, C775Y and/or S811F.

19. The method of any of claims 16-18, wherein the one or more EGFR exon 20 mutations comprise D770insNPG, S784F, R776C, S7681, V774M, S7681, H773insAH, H773insNPH, V774A, V769L, V769M, S768dupSVD, A763insLQEA, L792H, G796D, G796S, S784F, C775Y and/or S811F.

20. The method of any of claims 1-18, wherein the one or more EGFR TKI resistant mutations comprise one or more point mutations, insertions, and/or deletions of 3-18 nucleotides between amino acids 824-875 of exon 21.

21. The method of claim 20, wherein the one or more EGFR exon 21 mutations are located at one or more residues selected from the group consisting of L833, V834, G836, V843, T854, L861, L861, L862, L844 and L858.

22. The method of any of claim 20 or 21, wherein the one or more EGFR exon 21 mutations may comprise L833F, V834L, L858R, L861Q, V843I, L861R, L862V, L844V, L861Q, G836S, and/or T854I.

23. The method of any of claims 1-22, wherein the subject has been determined to have 2, 3, or 4 EGFR TKI resistant mutations.

24. The method of any one of claim 1-23, wherein the subject has been previously administered a TKI.

25. The method of claim 24, wherein the subject is resistant to the previously administered TKI.

26. The method of claim 24 or 25, wherein the TKI is lapatinib, erlotinib, gefitinib, rociletinib, olmutinib, naquotinib, osimertinib, ibrutinib, or nazartinib.

27. The method of any of claims 24-26, wherein the TKI is gefitinib, erlotinib, or osimeritinib.

28. The method of any of claims 24-27, wherein the TKI is osimeritinib.

29. The method of any of claims 1-28, wherein the one or more EGFR TKI resistant mutations are at residues E709, L718, G719, G724, C797, V843, T854, L861, and/or L792.

30. The method of any of claims 1-29, wherein the subject has been determined to not have an EGFR mutation at residue C797 or T790.

31. The method of any of claims 1-30, wherein the subject is determined to not have an EGFR mutation at residue T790.

32. The method of any of claims 1-29, wherein the subject has a T790 mutation.

33. The method of claim 32, wherein the subject has a T790 mutation in combination with at least one additional mutation.

34. The method of claim 31, wherein the subject is determined to have a mutation at residue at C797.

35. The method of any of claims 1-34, wherein the one or more EGFR TKI resistant mutations are selected from the group consisting of G719X, E709X, G724S, L718X, L861Q, T8541, V8431, C797S, and/or L792X, wherein X is any amino acid.

36. The method of any of claims 1-35, wherein the one or more EGFR TKI resistant mutations are selected from the group consisting of L861Q, G719S, L858R/L792H, L858R/C797S, and Exl9del/C797S.

37. The method of any of claims 1-36, wherein the subject was determined to have an EGFR TKI resistant mutation by analyzing a genomic sample from the patient.

38. The method of claim 37, wherein the genomic sample is isolated from saliva, blood, urine, normal tissue, or tumor tissue.

39. The method of any of claims 1-38, wherein the presence of an EGFR TKI resistant mutation is determined by nucleic acid sequencing or PCR analyses.

40. The method of any of claims 1-39, wherein the quinazolinamine derivative TKI is administered orally.

41. The method of any of claims 1-40, wherein the quinazolinamine derivative TKI is administered daily.

42. The method of claim 41, wherein the quinazolinamine derivative TKI is administered on a continuous basis.

43. The method of claim 42, wherein the quinazolinamine derivative TKI is administered on 28 day cycles.

44. The method of any of claims 1-43, further comprising administering an additional anti-cancer therapy.

45. The method of claim 44, wherein the additional anti-cancer therapy is chemotherapy, radiotherapy, gene therapy, surgery, hormonal therapy, anti-angiogenic therapy or immunotherapy.

46. The method of claim 44, wherein the quinazolinamine derivative TKI and/or anti-cancer therapy are administered intravenously, subcutaneously, intraosseously, orally, transdermally, in sustained release, in controlled release, in delayed release, as a suppository, or sublingually.

47. The method of claim 44, wherein administering the quinazolinamine derivative TKI and/or anti-cancer therapy comprises local, regional or systemic administration.

48. The method of claim 44, wherein the quinazolinamine derivative TKI and/or anti-cancer therapy are administered two or more times.

49. The method of any of claims 1-48, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

50. The method of any of claims 1-49, wherein the cancer is non-small cell lung cancer.

51. The method of any of claims 1-50, wherein the patient is human.

52. A pharmaceutical composition comprising quinazolinamine derivative TKI for use in a subject determined to have one or more EGFR TKI resistant mutations.

53. The composition of claim 52, wherein the composition is further defined as an oral composition.

54. The composition of claim 52 or 53, wherein the composition comprises 5-25 mg of a quinazolinamine derivative TKI.

55. The composition of any of claims 52-54, wherein the composition is formulated as a tablet.

56. The composition of any of claims 52-55, wherein the one or more EGFR TKI resistant mutations comprise a point mutation, insertion, and/or deletion of 1-18 nucleotides at exon 18, 19, 20, or 21.

57. The composition of any of claims 52-56, wherein the subject has been determined to have 2, 3, or 4 EGFR TKI resistant mutations.

58. The composition of any of claims 52-57, wherein the one or more EGFR TKI resistant mutations are at residues E709, L718, G719, G724, C797, V843, T854, L861, and/or L792.

59. The composition of any of claims 52-58, wherein the subject has been determined to not have an EGFR mutation at residue C797 or T790.

60. The composition of any of claims 52-59, wherein the one or more EGFR TKI resistant mutations are selected from the group consisting of G719X, E709X, G724S, L718X, L861Q, T8541, V8431, C797S, and/or L792X, wherein X is any amino acid.

61. The composition of any of claims 52-59, wherein the one or more EGFR TKI resistant mutations are selected from the group consisting of L861Q, G719S, L858R/L792H, L858R/C797S, and Exl9del/C797S.

62. The composition of any of claims 52-61, wherein the subject is being treated with an anti-cancer therapy.

63. A method of predicting a response to a quinazolinamine derivative TKI alone or in combination with a second anti-cancer therapy in a subject having a cancer comprising detecting a EGFR TKI resistant mutation in a genomic sample obtained from said patient, wherein if the sample is positive for the presence of the EGFR TKI resistant mutation, then the patient is predicted to have a favorable response to the quinazolinamine derivative TKI alone or in combination with an anti-cancer therapy.

64. The method of claim 63, wherein the EGFR TKI resistant mutation is at residue E709, L718, G719, G724, C797, V843, T854, L861, and/or L792.

65. The method of claim 63 or 64, wherein the genomic sample is isolated from saliva, blood, urine, normal tissue, or tumor tissue.

66. The method of any of claims 63-65, wherein the presence of a HER exon 21 mutation is determined by nucleic acid sequencing or PCR analyses.

67. The method of any of claims 63-66, wherein the EGFR TKI resistant mutation is selected from the group consisting of G719X, E709X, G724S, L718X, L861Q, T8541, V8431, C797S, and/or L792X, wherein X is any amino acid.

68. The method of any of claims 63-67, wherein the EGFR TKI resistant mutation is selected from the group consisting of L861Q, G719S, L858R/L792H, L858R/C797S, and Exl9del/C797S.

69. The method of any of claims 63-68, wherein a favorable response to the quinazolinamine derivative TKI alone or in combination with an anti-cancer therapy comprises reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, or increased patient survival.

70. The method of any of claims 63-69, further comprising administering the quinazolinamine derivative TKI alone or in combination with a second anti-cancer therapy to said patient predicted to have a favorable response.

71. The method of any of claims 63-70, wherein the quinazolinamine derivative TKI is administered orally.

72. The method of any of claims 63-71, wherein the quinazolinamine derivative TKI is administered at a dose of 5-25 mg.

73. The method of any of claims 63-72, wherein the quinazolinamine derivative TKI is formulated as a tablet.


SEQUENCE LISTING (1 sequence)

SEQ ID NO: 1 - DNA, 13 bp, Artificial sequence
  Synthetic oligo
gcaacatctccgg